(19) United States
(12) Patent Application Publication
(10) Pub. No.: US 2026/0366079 A1
(43) Pub. Date: Nov. 19, 2026

(54) HLA Haplotypes and Uses Thereof

(71) Applicant: University of Florida Research Foundation, Inc., Gainesville, FL (US)

(72) Inventors: Meghan A. Berryman, Gainesville, FL (US); Eric W. Triplett, Gainesville, FL (US)

(21) Appl. No.: 19/139,563

(22) PCT Filed: Dec. 21, 2023
(86) PCT No.: PCT/US2023/085301

§ 371 Date: Jun. 16, 2025

Related U.S. Application Data
(60) Provisional application No. 63/476,653, filed on Dec. 22, 2022.

(51) Int. Cl. C12Q 1/6883; A61K 35/00; A61K 35/745

(52) U.S. Cl. CPC C12Q 1/6883; A61K 35/745; A61K 2035/115; C12Q 2600/156; C12Q 2600/172

(57) ABSTRACT

The disclosure provides a method comprising (a) identifying the presence of a protective HLA haplotype allele in a sample from a subject suffering from an autoimmune disorder; and (b) administering a composition comprising Bifidobacterium genus probiotic to the subject. Also provided is a method comprising (a) identifying the presence of a non-protective HLA haplotype allele in a sample from a subject; and (b administering a composition comprising a Bifidobacterium selected from B. longum, B. breve and B. pseudocatenulatum to the subject.

### INCORPORATION BY REFERENCE OF MATERIAL SUBMITTED ELECTRONICALLY

Incorporated by reference in its entirety is a computer-readable nucleotide/amino acid sequence listing submitted concurrently herewith and identified as follows: file name; “57822_SeqListing.XML,” 6,136 bytes, created on Dec. 20, 2023.

### BACKGROUND

Class II HLA proteins are major players in cell-mediated adaptive immune response [7]. They are composed of an alpha and beta chain and are encoded by three pairs of highly polymorphic genes, HLA-DR, HLA-DQ, and HLA-DP. The extracellular regions form an immunoglobulin-like domain and a peptide-binding cleft, where the most polymorphic sites of HLA class II molecules are located. Polymorphisms within these haplotypes cause structure-altering changes to HLA class II proteins at the amino acid level [8-11]. These changes alter peptide-binding capabilities of HLA and thus change the antigen presenting capabilities [7,12,13]. HLA class II proteins are expressed in intestinal epithelial cells at a steady state in the presence of a healthy gut microbiome, but dysbiosis and inflammation increases intestinal epithelial HLA class II expression [14,15]. Allele-specific binding pockets along the peptide-binding cleft of HLA class II molecule interact with antigen anchoring peptides. This gives rise to the potential for HLA polymorphisms to affect peptide binding and presentation [16]. The structural differences between HLA haplotype proteins, specifically HLA-DR and HLA-DQ, result in unique sensitivities and can be the determining factor for many autoimmune diseases, such as type 1 diabetes (T1D), celiac disease (CD), rheumatoid arthritis (RA), and inflammatory bowel diseases (IBD) including ulcerative colitis (UC) and Crohn's disease (CrD) [8,17-19].

### SUMMARY

The disclosure provides a method comprising identifying the presence of a protective HLA haplotype allele in a sample from a subject and administering a Bifidobacterium genus probiotic to the subject.

### BRIEF DESCRIPTION OF THE FIGURES

FIG. 1. Relative abundance correlation trends for Bifidobacterium genus with haplotype dosage. P-values for Wilcoxon signed-rank test are listed.

FIG. 2. Significant differential abundance in Bifidobacterium ASVs and HLA dosage. Log fold change correlation between ASVs and HLA haplotype dosage. DESeq defined padj<0.05. Positive log fold change is associated with the first dosage indicated in the panel.

FIG. 3. Cohen's d effect size of HLA haplotype dosage on Bifidobacterium relative abundance. An effect size of 0.2 is a small effect, 0.5 is a medium effect, and 0.8 is a large effect. Points above 0 are showing correlation with the dosage of the right. Points below 0 are showing correlation with dosage on the left. Points outside the dotted lines have significant effect.

### DETAILED DESCRIPTION

The present disclosure is based, at least in part, on the discovery that the presence of certain human leukocyte antigen (HLA) alleles in a subpopulation of subjects having certain HLA alleles experience benefits associated with a Bifidobacterium probiotic. Bifidobacterium may be from species such as B. adolescentis, B. animalis, B. animalis subsp. animalis, B. animalis subsp. lactis, B. bifidum, B. breve, B. catenulatum, B. longum, B. longum subsp. infantis, B. longum subsp. longum, B. pseudocatenulatum and B. pseudolongum. In some embodiments, the Bifidobacterium is B. pseudocatenulatum, B. bifium, B. breve and B. longum.

In one aspect, described herein is a method comprising identifying the presence of a protective HLA haplotype allele in a sample from a subject and administering a composition comprising a Bifidobacterium to the subject. In some embodiments, the subject is suffering from an autoimmune disorder. In some embodiments, the subject is at risk of developing an autoimmune disorder. In some embodiments, the autoimmune disorder is ulcerative colitis, Crohn's disease, irritable bowel syndrome, type 1 diabetes, celiac disease, or rheumatoid arthritis.

In some embodiments, the protective HLA haplotype allele comprises HLA-DR15/DQ6.2

In another aspect, the disclosure provides a method comprising identifying the presence of a non-protective HLA haplotype allele in a sample from a subject and administering a composition comprising a Bifidobacterium selected from B. longum, B. breve and B. pseudocatenulatum to the subject. In some embodiments, the subject is suffering from an autoimmune disorder. In some embodiments, the subject is at risk of developing an autoimmune disorder.

In some embodiments, the subject is human. In some embodiments, the subject is an infant (e.g., less than 12 months in age). In some embodiments, the subject is a child (i.e., ≥12 months and <18 years in age). In some embodiments, the subject is an adult (≥18 years in age).

### Detection of HLA Alleles

Detection of HLA alleles can be used to determine whether a subject, such as, for example, a human subject, will respond to administration of a Bifidobacterium strain. Assays such as RT-PCR, PCR, qPCR, DNA and RNA sequencing, microarray analysis and any other genome-based analyses known in the art, along with any suitable immunoassay, may be used to detect an HLA allele described herein in a sample from the subject, for example, stool, plasma, serum, cerebrospinal fluid, sputum, saliva, breast milk, tears, bile, semen, vaginal secretion, amniotic fluid, urine, stool, leukocytes, bone marrow cells, buccal cells, fibroblasts and lung or other tissue biopsies. In some embodiments, the sample is a stool sample. In addition, such analyses may be qualitative or quantitative.

Subjects may be screened for the presence of an HLA allele through the use of quantitative reverse transcriptase-polymerase chain reaction (qRT-PCR), which utilizes competitive techniques employing an internal homologous control that differs in size from the target, for example, by a small insertion or deletion. Non-competitive and kinetic quantitative PCR or RT-PCR may also be used. Experiments may combine real-time, kinetic PCR or RT-PCR detection together with an internal homologous control that can be simultaneously detected alongside the target sequences. In one non-limiting embodiment, real time quantitative PCR may provide the capability of measuring the level of an HLA allele gene product amplified through PCR. Quantitative PCR may require only a nominal amount of a sample to perform such experiments.

Quantitative amplification is based on the monitoring of a signal (e.g., fluorescence of a probe) representing copies of a template in cycles of an amplification (e.g., PCR) reaction. In the initial cycles of the PCR, a very low signal is observed because the quantity of the amplification product formed does not support a measurable signal output from the assay. After the initial cycles, as the amount of formed amplification product increases, the signal intensity increases to a measurable level and reaches a plateau in later cycles when the PCR enters into a non-logarithmic phase. Through a plot of the signal intensity versus the cycle number, the specific cycle at which a measurable signal is obtained from the PCR reaction can be deduced and used to back-calculate the quantity of the target before the start of the PCR. The number of the specific cycles that is determined by this method is typically referred to as the cycle threshold (Ct). Exemplary methods are described in, e.g., U.S. Pat. Nos. 6,180,349; 6,033,854; and 5,972,602.

In some embodiments, nucleic acid microarrays, or gene chip technology, may be used to screen and identify subjects having a protective (or non-protective) HLA haplotype allele. A “microarray” is an array of distinct polynucleotides, oligonucleotides, polypeptides, peptides, or antibodies affixed to a substrate, such as paper, nylon, or other type of membrane; filter; chip; glass slide; or any other type of suitable support.

In some embodiments, the microarray technology involves the positioning of highly condensed and ordered arrays of nucleic acid probes, for example, DNA oligonucleotides, on a substrate, for example, a glass slide or nylon membrane. Each oligonucleotide may comprise a nucleotide sequence that is complementary to a portion of an HLA allele, wherein the oligonucleotide can be placed on a single glass slide or nylon membrane. For example, and not by way of limitation, up to 50,000 DNA fragments, may be placed on a single glass slide and up to 5,000 placed on a nylon membrane. The resulting microarrays can then be used to screen for the presence an HLA allele transcription product expressed in a sample to be screened.

In some embodiments, a nucleic acid microarray may be utilized by preparing labeled nucleic acid from a sample to be screened and hybridized such labeled nucleic acid with the array. In addition, labeled nucleic acid of a designated control sequences may be prepared (or in the event that the array is sold as part of a kit, could be supplied to the user). Radioactive, colorimetric, chemiluminescent or fluorescent tags may be used for labeling of nucleic acid sequences from the sample and for the control. Numerous techniques for scanning arrays, detecting fluorescent, chemiluminescent, or colorimetric output, are known in the art and may be used for detecting hybridization of a nucleic acid from a test sample to the microarray. For example, a low-cost, high-throughput fluorescent microarray scanning system (ScanArray®, PerkinElmer Life And Analytical Sciences, Inc., Waltham, MA, USA), or a colorimetric microarray scanner (Arraylt® SpotWare™, TeleChem International, Inc., Sunnyvale, CA, USA) may be used. Numerous protocols for the preparation of labeled nucleic acid sequences are publicly available and may be used.

In some embodiments, the identifying step comprises screening for an HLA allele through sequencing (i.e. determining the nucleotide sequence of a given DNA or RNA fragment) of a genomic DNA or HLA allele expression product present in a sample taken from the subject. Any sequencing methods known in the art may be used to determine the nucleotide order of the HLA allele DNA or RNA. For example, but not by way of limitation, an HLA allele may be identified by first performing PCR and then sequencing the product of PCR to determine the specific allele (see Listgarten et al., 2008, PloS omput. Biol. 4(2):e 1000016).

The sequencing of a nucleic acid sample (i.e., determining the nucleotide order of a given DNA or RNA fragment) is not limited to any one technique. The present invention contemplates the use of any sequencing technique known in the art.

In some embodiments, the identifying step comprises the use of antibodies in an immune assay to detect protein isoforms resulting from expression of a protective (and optionally non-protective) HLA haplotype allele in a sample from the subject. Exemplary immunoassays include, but are not limited to, enzyme linked immunosorbent assays (ELISAs), Western blots and radioimmunoassays (RIA).

In some embodiments, the subject is homozygous for the protective HLA haplotype allele. In some embodiments, the method comprises identifying that the subject is homozygous for HLA-DR1/DQ5. In some embodiments, the method comprises identifying that the subject is homozygous for HLA-DR15/DQ6.2. In some embodiments, the method comprises administering B. pseudocatenulatum ASV 1638 (SEQ ID NO: 1) to the subject.

In some embodiments, the method further comprises identifying the presence of a non-protective HLA haplotype allele in a sample from the subject. In some embodiments, the non-protective HLA haplotype allele comprises HLA-D3/DQ2.5 or HLA-DR4/DQ8.

In some embodiments, the subject is heterozygous for the protective HLA haplotype allele. In some embodiments the method comprises identifying that the subject is heterozygous for (a) HLA-DR1/DQ5 and HLA-D3/DQ2.5, or (b) HLA-DR1/DQ5 and HLA-DR4/DQ8.

In some embodiments, the non-protective HLA haplotype allele comprises HLA-D3/DQ2.5 or HLA-DR4/DQ8. In some embodiments, the subject is homozygous for HLA-DR3-DQ2 or HLA-DR4-DQ8. In some embodiments, the subject is homozygous for HLA-DR3-DQ2. In some embodiments, the method comprises administering a composition comprising B. longum ASV1491 (SEQ ID NO: 2) to the subject.

In some embodiments, the method comprises identifying the presence of HLA-DR3-DQ2/DR3-DQ2 allele in a sample from the subject and administering a composition comprising B. longum ASV1491 (SEQ ID NO: 2) to the subject.

In some embodiments, the method comprises identifying the presence of HLA-DR4-DQ8/DR4-DQ8 allele in a sample from the subject and administering a composition comprising B. breve ASV1485 (SEQ ID NO: 3) to the subject.

In some embodiments, the method comprises identifying the presence of HLA-DR1-DQ5/DR1-DQ5 allele in a sample from the subject and administering a composition comprising B. bifidum ASV1482 (SEQ ID NO: 4) to the subject.

### Compositions and Routes of Administration

The Bifidobacteria described herein can be for any suitable route of administration. For example, the Bifidobacteria is formulated into a composition that can be administered to the subject via oral administration, rectal administration, transdermal administration, intranasal administration or inhalation. In some embodiments, the probiotic is administered to the subject orally.

Suitable oral dosage forms for the compositions described herein include tablets, capsules, solutions, suspensions, syrups, lozenges, and dry powders. Tablets can be made using compression or molding techniques well known in the art. Gelatin or non-gelatin capsules can prepared as hard or soft capsule shells, which can encapsulate liquid, solid, and semi-solid fill materials, using techniques well known in the art.

In some embodiments, the composition further comprises an acceptable carrier. The term “carrier” includes, but is not limited to, lipids, phospholipids, salts, emulsifiers, excipients, diluents, preservatives, binders, lubricants, disintegrators, swelling agents, fillers, stabilizers, and combinations thereof. Polymers used in the dosage form include hydrophobic or hydrophilic polymers and pH dependent or independent polymers. Exemplary hydrophobic and hydrophilic polymers include, but are not limited to, hydroxypropyl methylcellulose, hydroxypropyl cellulose, hydroxyethyl cellulose, carboxy methylcellulose, polyethylene glycol, ethylcellulose, microcrystalline cellulose, polyvinyl pyrrolidone, polyvinyl alcohol, polyvinyl acetate, and ion exchange resins. “Carrier” also includes all components of the coating composition, which may include plasticizers, pigments, colorants, stabilizing agents, and glidants.

The compositions described herein can be prepared using one or more excipients, including diluents, preservatives, binders, lubricants, disintegrators, swelling agents, fillers, stabilizers, and combinations thereof.

In some embodiments, the composition comprises at least about 10⁴ colony forming units (cfu) of a Bifidobacterium. In some embodiments, the composition comprises at least about 10⁴, 10⁵, 10⁶, 10⁷, 10⁸, 10⁹, 10¹⁰, 10¹¹, 10¹², or 10¹³ cfu, including ranges between any of the listed values, for example 10⁴-10⁸ cfu, 10⁴-10⁹ cfu, 10⁴-10¹⁰ cfu, 10⁴-10¹¹ cfu, 10⁴-10¹² cfu, 10⁴-10¹³ cfu, 10⁵-10⁸ cfu, 10⁵-10⁹ cfu, 10⁵-10¹⁰ cfu, 10⁵-10¹¹ cfu, 10⁵-10¹² cfu, 10⁵-10¹³ cfu, 10⁶-10⁸ cfu, 10⁶-10⁹ cfu, 10⁶-10¹⁰ cfu, 10⁶-10¹¹ cfu, 10⁶-10¹² cfu, 10⁶-10¹³ cfu, 10⁷-10⁸ cfu, 10⁷-10⁹ cfu, 10⁷-10¹⁰ cfu, 10⁷-10¹¹ cfu, 10⁷-10¹² cfu, 10⁷-10¹³ cfu, 10⁸-10⁹ cfu, 10⁸-10¹⁰ cfu, 10⁸-10¹¹ cfu, 10⁸-10¹² cfu, or 10⁸-10¹³ cfu of a Bifidobacterium.

In one aspect, described herein is a method of preparing a probiotic composition for a subject in need thereof comprising (a) identifying the presence of a HLA-D3-DQ2/DR2-DQ2 haplotype allele in a sample from the subject and (b) combining B. longum with a carrier, excipient or diluent.

In another aspect, described herein is a method of preparing a probiotic composition for a subject in need thereof comprising (a) identifying the presence of a HLA-D4-DQ8/HLA-DR4-DQ8 HLA haplotype allele in a sample from the subject and (b) combining B. breve with a carrier, excipient or diluent.

In another aspect, described herein is a method of preparing a probiotic composition for a subject in need thereof comprising (a) identifying the presence of a HLA-DR15-DQ6.2/HLA-DR15-DQ6.2 HLA haplotype allele in a sample from the subject and (b) combining B. pseudocatenulatum with a carrier, excipient or diluent.

In another aspect, described herein is a method of preparing a probiotic composition for a subject in need thereof comprising (a) identifying the presence of a HLA-DR1-DQ5/HLA-DR1-DQ5 HLA haplotype allele in a sample from the subject and (b) combining B. faecale with a carrier, excipient or diluent.

In any of the methods of preparing a probiotic composition described herein, the method may further comprise administering the composition to a subject or ingestion by the subject.

In some embodiments, the compositions described herein is incorporated into a food prodsuct. The term “food product” as used herein refers to any substance containing nutrients that can be ingested by an organism to produce energy, promote health and wellness, stimulate growth, and maintain life. Exemplary food products include, but are not limited to, baked goods (cakes, cookies, crackers, breads, scones and muffins), dairy-type products (including but not limited to cheese, yogurt, custards, rice pudding, mousses, ice cream, frozen yogurt, frozen custard), desserts (including, but not limited to, sherbet, sorbet, water-ices, granitas and frozen fruit purees), spreads/margarines, pasta products and other cereal products, meal replacement products, nutrition bars, trail mix, granola, beverages (including, but not limited to, smoothies, water or dairy beverages and soy-based beverages), and breakfast type cereal products such as oatmeal.

In some embodiments, the food product is a meal replacement product. The term “meal replacement product” as used herein refers to a food product that is intended to be eaten in place of a normal meal. Nutrition bars and beverages that are intended to constitute a meal replacement are types of meal replacement products. The term also includes products which are eaten as part of a meal replacement weight loss or weight control plan, for example snack products which are not intended to replace a whole meal by themselves, but which may be used with other such products to replace a meal or which are otherwise intended to be used in the plan. These latter products typically have a calorie content in the range of from 50-200 kilocalories per serving.

All of the U.S. patents, U.S. patent application publications, U.S. patent applications, foreign patents, foreign patent applications and non-patent publications referred to in this specification and/or listed in the Application Data Sheet, are incorporated herein by reference, in their entireties.

From the foregoing it will be appreciated that, although specific embodiments of the invention have been described herein for purposes of illustration, various modifications may be made without deviating from the spirit and scope of the invention.

### Examples

### Materials and Methods:

HLA Genotyping: HLA DR-DQ haplotypes were defined using typing for DQB1 alleles and informative DQA1 alleles for deducing the common European DR-DQ haplotypes. Typing was performed using hybridization with sequence-specific lanthanide-labelled oligonucleotide probes [23]. HLA genotypes were included from all available blood spots in ABIS children. The dosage counts for HLA haplotypes of interest are provided in Table 2 (below). The identified HLA DR-DQ haplotypes and corresponding confirmed alleles present (in parentheses) are as follows: DR1-DQ5 (DQB1*05:01); DR3-DQ2.5, (DQA1*05, DQB1*02); DR4-DQ7 (DQA1*03, DQB1*03:01); DR4-DQ8 (DQA1*03, DQB1*03:02); DR7-DQ2 (DQA1*02:01, DQB1*02), DR7-DQ9 (DQA1*02:01, DQB1*03:03), DR8-DQ4 (DQB1*04); DR9-DQ9 (DQA1*03, DQB1*03:03); DR13-DQ6.3 (DQB1*06:03); DR13-DQ6.4 (DQB1*06:04); DR15-DQ6.2 (DQB1*06:02).

The highest genetic risks for developing autoimmunity are considered to be those homozygous for DR3-DQ2.5 or DR4-DQ8 or a heterozygous combination of the two. The most protective haplotype against developing autoimmunity is DR15-DQ6.2. HLA haplotype DR1-DQ5 has generally been regarded as a neutral haplotype.

Processing Amplicon Sequence Variants: DNA extraction, 16S rRNA barcoded PCR, and V3-V4 16S rRNA Illumina sequencing were performed on samples collected at 1 year of age as previously described[1]. Bacterial quantification through universal 16S rRNA primers was performed as previously described [24]. Paired-end reads were merged, primers were removed, and amplicon sequence variant (ASV) processing were performed as previously described [25]. Taxonomy was assigned using the SILVA_v138 database [26,27]. Species not determined through SILVA_v138 were manually input using BLAST and the rRNA database. Only species higher than 99.27% percent identity were added. Subspecies were not included.

Of the original 1,756 one-year-old infants, filtering excluded samples with fewer than 1,000 total reads and incomplete HLA genotype information, leaving 1,326 infant samples. ASVs under five reads in fewer than five subjects after using the filter_taxa function from phyloseq R package were excluded, resulting in 539 ASVs [28]. The average read count for the 1,326 infant samples analyzed was 67,756 and the maximum read count was 606,188. Relative abundance of samples was determined with transform_sample_counts, and the reads/g was calculated by multiplying the relative abundance and copies of 16S rRNA per gram of stool found from quantitative polymerase chain reaction (qPCR). ASVs were conglomerated into genera using the tax_glom function from the phyloseq package. The genera were passed through phyloseq_to_deseq, then the estimateSizeFactors using the “poscounts” format to allow for samples containing zero [29].

Cohort Matching: Matching was performed based on total breastfeeding,siblings at birth, and single mother according to haplotype dosage (Krusal-Wallis: FDR<0.05). Individuals homozygous for a specific haplotype were matched on these criteria to individuals with 0 instances of that haplotype to create the matched 0 dosage group. Homozygous individuals were then matched on the same criteria to individuals that were heterozygous for that haplotype, creating the matched 1 dosage group. The dosage counts for HLA haplotypes of interest before and after matching are provided in Table 2 (below).

Statistical Analysis: Factors impacting binomial beta diversity were tested for using the permutational multivariate analysis of variance (PERMANOVA) test through the adoniso function in the vegan R package with 999 permutations [29]. P-values were corrected for false discovery rate (FDR) using the Benjamin-Hochberg method.

Core microbiomes were determined through the R package PIME [30]. The associated principle coordinate analysis (PCoA) plot was created through the plot_ordination(function.

Relative abundances were calculated using the transform_sample_counts function. To calculate total abundance, the relative abundance values were multiplied by sample by the total number of copies of 16s rRNA per gram of stool, as determined through qPCR.

Heatmaps were calculated using the above relative abundance and reads/gram methods. Prevalence is defined here as the percent of individuals in the cohort with non-zero abundance of the respective ASV or taxa. Heatmaps were designed using the R package ‘pheatmap’ [31]

Boxplots were designed using the R package ggplot2 [32]. The function stat_compare_means was used to determine Kruskal-Wallis, Wilcoxon, and T-test p-values. The function ggarrange was used to arrange each graph into a figure.

Cohen's d effect size is a representation of the standardized mean difference. Effect size was calculated and graphed in Microsoft Excel [32]. To calculate the standardized mean difference, the difference between the mean relative abundance of each ASV or taxa within two haplotype dosages were divided by the standard deviation of the population sampled.

Differential abundances of microbes without a prevalence threshold were assessed with the binomial distribution model R package, DESeq2 [34]. The estimateSizeFactor function was used with “poscount” type, allowing for samples absent of the respective taxas. Within DESeq2, the Wald test for calculating p values, with a local fit type, was used. The additional Cook's distance filtering was not considered. Differential abundance graphs were designed with the function ggplot [31].

### Example 1—Meta-Analysis of Probiotic Clinical Trials

To gain perspective on the clinical effectiveness of probiotic supplementation on autoimmune diseases, a meta-analysis of randomized controlled clinical trials of well-defined probiotics in patients diagnosed with autoimmune diseases was performed (Table 1).

TABLE 1
Randomized Controlled Clinical Trials of Well-Defined
Probiotics in Patients Diagnosed with an Autoimmune Disease.

| | | | Total | | | |
|---|---|---|---|---|---|---|
| | | | Participants | | | |
| Disease | Probiotic | Duration | (control) | Genetics | Measures | Results |
| T1D | L. rhamnosus GG | 6 months | 96 (48) | No | C-peptide | NS |
| [34-36] | | | | | Cytokines | |
| | B. lactis Bb12 | | | | Zonulin level | NS |
| | | | | | Insulin req. | NS |
| | | | | | | NS |
| | L. rhamnosus GG | 6 months | 64 (30) | No | QIV resp. | NS |
| | | | | | Cytokine resp. | Decreased IFN-γ *** |
| | | | | | | & IL-17F ** response |
| | L. rhamnosus GG | 6 months | 14 (7) | No | T1D diagnosis | NS |
| | L. rhamnosus LC705 | | | | Autoantibody | NS |
| | B. breve Bb99 | | | | acquisition | |
| | P. freudenreichii | | | | | |
| | subsp. shermanii JS | | | | | |
| CD | L. plantarum HEAL9 | 6 months | 78 (38) | Yes- | Lymphocytes | NS |
| [37-42] | L. paracasei 8700:2 | | | MHCII | IgA-tTG | NS-Decreased |
| | | | | | | in both groups |
| | | | | | IgG-tTG | NS-Decreased |
| | | | | | | in both groups |
| | B. infantis NLS-SS | 8 weeks | 12 (7) | No | CSI | Decreased only in |
| | | | | | | severe CD patients ** |
| | | | | | GIP | NS |
| | L. casei LMG | 6 weeks | 109 (55) | No | IBS-SSS | Decreased with |
| | 101/37 P-17504 | | | | | probiotic *** |
| | L. plantarum CECT 4528 | | | | GSRS | Decreased with |
| | | | | | | probiotic *** |
| | B. animalis subsp. lactis | | | | BSFS | NS |
| | Bil LMG P-17502 | | | | IBS-QOL | NS |
| | B. breve Bbr8 LMG P-17501 | | | | | |
| | B. breve B110 LMG P-17500 | | | | | |
| | L. rhamnosus GG | 6 months | 871 (439) | Yes- | CD diagnosis CD | NS |
| | L. rhamnosus LC705 | | | MHCII | antibodies | NS |
| | B. breve Bb99 | | | | | |
| | P. freudenreichii | | | | | |
| | subsp. shermanii JS | | | | | |
| | B. breve BR03 | 3 months | 46 (24) | No | IL-10 | NS |
| | B. breve B632 | | | | TNF-α | NS-Decreased with |
| | | | | | | probiotic compared to |
| | | | | | | baseline but not placebo ** |
| | B. longum CECT 7347 | 3 months | 33 (16) | No | CD3⁺ | Decreased with probiotic ** |
| | | | | | Lymphocytes | NS-HLA-DR+, CD4+, |
| | | | | | | CD4+Foxp3+, CD8+ |
| | | | | | TNF-α | Decreased |
| | | | | | | with probiotic* |
| | | | | | Cytokines | NS-IFN-γ, IL-10, |
| | | | | | | IL-13, TGF-ß1 |
| | | | | | Serum IgG | NS |
| | | | | | Fecal sIgA | Decreased |
| | | | | | | with probiotic ** |
| | | | | | CD Symptoms | NS |
| IBD | L. rhamnosus NCIMB 30174 | 4 weeks | UC 81 (40) | No | IBS-QOL | NS |
| [43-50] | L. plantarum NCIMB 30173 | | | | CDA | NS |
| UC | L. acidophilus NCIMB 30175 | | CrD 62 (29) | | FCAL | Decreased with |
| &/or | | | | | | probiotic only in UC* |
| CrD | E. faecium NCIMB 30176 | | | | Laboratory | NS-Hgb, HCT, |
| | | | | | | WBC, ESR, CRP |
| | E. faecium | 8 weeks | UC 36 (18) | No | CRP | Decreased |
| | (no strains specified) | | | | | with probiotic ** |
| | L. plantarum | | | | | NS-Hgb, WBC, |
| | | | | | | lymphocyte, neutrophil |
| | | | | | | NS-Decreased |
| | | | | | | in both groups |
| | S. thermophilus | | | | Laboratory | More change |
| | B. lactis | | | | | with probiotic ** |
| | L. acidophilus | | | | UCEIS | |
| | B. longum | | | | TWCAI | |
| | B. breve Yakult | 48 weeks | UC 195 (97) | No | Relapse-free | NS |
| | L. acidophilus | | | | survival | NS |
| | | | | | Time to relapse | |
| | L. salivarius | 24 months | UC 60 (30) | No | MMDAI | Decreased more |
| | | | | | | with probiotic at |
| | L. acidophilus | | | | | all time points ** |
| | B. bifidum BGN4 | | | | Stool frequency | Decreased |
| | | | | | Endoscopy | with probiotic |
| | | | | | | at 6 and 24 months ** |
| | | | | | Rectal bleeding | More improvement |
| | | | | | | after 18 months with |
| | | | | | | probiotic *** |
| | | | | | | Decreased |
| | | | | | | with probiotic |
| | | | | | | at 6, 18, and 24 months ** |
| | B. longum 536 | 8 weeks | UC 47 (23) | No | UCDAI | NS-Decreased with |
| | | | | | Endoscopy | probiotic compared to |
| | | | | | | baseline but not placebo ** |
| | | | | | Remission | NS |
| | | | | | | NS |
| | E. faecalis T-110 | 12 months | UC 46 (23) | No | Relapse | NS |
| | C. butyricum TO-A | | | | Remission | NS |
| | B. mesentericus TO-A | | | | | |
| | B. infantis 35624 | 6 weeks | UC 22 (9) | No | TNF-α | NS |
| | | | | | IL-6 | NS-Decreased with |
| | | | | | CRP | probiotic compared to |
| | | | | | | placebo but not baseline* |
| | | | | | | NS |
| | L. acidophilus La-5 | 52 weeks | UC 22 (12) | No | Relapse | NS |
| | B. animalis | | | | Remission | NS |
| | subsp. lactis BB-12 | | | | | |
| RA | L. acidophilus La-14 | 60 days | 42 (21) | No | WBC | Decreased |
| [51,52] | | | | | | with probiotic ** |
| | L. casei Lc-11 | | | | TNF-α | NS-Decreased |
| | | | | | IL-6 | in both groups |
| | L. lactis LI-23 | | | | IL-10 | Decreased |
| | | | | | | with probiotic ** |
| | B. lactis B1-04 | | | | Adiponectin | NS |
| | B. bifidum Bb-06 | | | | CRP | NS |
| | | | | | ESR | NS |
| | | | | | | NS |
| | | | | | Ferritin | NS |
| | | | | | DAS-28 | NS |
| | B. coagulans | 60 days | 44 (22) | No | HAQ-DI | NS |
| | GBI-30, 6086 | | | | Pain Scale | Decreased mean |
| | | | | | | with probiotic ** |
| | | | | | ESR | NS |
| | | | | | CRP | NS |

*Abbreviations as follows: Quadrivalent inactivated influenza vaccination (QIV), tissue transglutaminase (tTG), celiac symptom index (CSI), gluten immunogenic peptide (GIP), IBS Severity Scoring System (IBS-SSS), Gastrointestinal Symptom Rating Scale (GSRS), Bristol Stool Form Scale (BSFS), and IBS Quality of Life Questionnaire (IBS-QOL), clinical disease activity (CDA), fecal calprotectin (FCAL), ulcerative colitis endoscopic index of severity (UCEIS), Truelove-Witts Clinical Activity Index (TWCAI), erythrocyte sedimentation rate (ESR), Modified Mayo Disease Activity Index (MMDAI), Ulcerative Colitis Disease Activity Index (UCDAI), C-reactive protein (CRP), erythrocyte sedimentation rate (ESR), Disease Activity Score-28 (DAS-28), Stanford Health Assessment Questionnaire Disability Index (HAQ-DI). * p < 0.1; ** p < 0.05; *** p< 0.01

Of the 50 genera used in the examined trials, Lactobacillus and Bifidobacterium made up 82% of probiotic intervention, 42% and 40% respectively. The remaining genera were Bacillus (2%), Propionibacterium (4%), Enterococcus (6%), Streptococcus (2%), Lactococcus (2%), and Clostridium (2%). The most predominant species studies were Lactobacillus rhamnosus (14%), Lactobacillus acidophilus (12%), and Bifidobacterium breve (14%).

Three clinical trials focusing on the effect of probiotics on T1D were published over the past 10 years [34-36]. Two of the studies were on multi-strain supplementation. All T1D studies included L. rhamnosus GG. Of the three clinical trials, the only significant influence seen from probiotic supplementation was the reduction of IFN-γ & IL-17F levels compared to placebo in response to quadrivalent inactivated influenza vaccination in activated peripheral blood mononuclear cells of children and adolescents with T1D. However, overall humoral response between probiotic and control were insignificant [36]. The predominant result from T1D probiotic clinical trials was no significant difference between treatment and placebo.

Six clinical trials studying the effect of probiotics on CD symptom relief were published within the past 10 years [37-42]. Four of the studies were on multi-strain supplementation and half of the studies included B. breve strains. Two of the six studies showed survey-indicated relief of CD symptoms, one of which only found a decrease in symptoms in patients with the most severe CD at baseline [38,39]. Only one study observed a change in cytokine levels with a moderately significant decrease in TFN-α compared to placebo (p=0.067) [42]. That same study was also the only trial to show that Bifidobacterium longum CECT 7347 supplementation changed the lymphocyte population with a significant decrease in CD3+ T-cells when compared to placebo (p=0.020) [42]. The predominant result from CD probiotic clinical trials was no significant improvement in CD symptomology with probiotic treatment.

Eight clinical trials on the effect of probiotics on IBD were published since 2012 [43-50]. Lactobacillus acidophilus was included in all but one of the six multi-strain supplementation studies. However, half of the studies did not specify strains for all the bacterial species tested. The predominant result from IBD probiotic clinical trials was no significant, consistent change in symptomology between treatment and placebo measurements. Although Groeger et al. showed Bifidobacterium infantis 35624 intervention in healthy subjects to reduce both TNF-α and IL-6, no evidence for the effectiveness of mitigation in UC was found [49].

Two clinical trials on the effect of probiotics on rheumatoid arthritis (RA) symptomology within the past 10 years met our standard for analysis. Most measured factors resulted in non-significant changes. Bifidobacterium coagulans GBI-30 supplementation decreased pain, white blood cell count, and IL-6 in a multi-strain probiotic study [51,52].

Of the 19 probiotic supplementation trials conducted on autoimmunity, only two considered the genetic makeup of the subjects, but none of the studies controlled for genetics.

### Example 2—Description of Cohort, Role of HLA, and Confounding Variables

ABIS is a prospective general-population cohort of 17,055 children born in southeast Sweden between Oct. 1, 1997 and Oct. 1, 1999 [22]. ABIS collected biological specimens longitudinally from birth, 1 year, 2-3 years, 5-6 years, and 11-13 years of age, creating a large biobank of stool, urine, blood, and hair. Metadata was acquired in the form of diary and HLA genotyping. Collected information included but was not limited to infection history, antibiotic use, duration of breastfeeding, introduction to or frequency of certain food consumption, living conditions and additional environmental factors. Following filtering, 1,326 infant samples were analyzed. Here each ASV is considered to be a different bacterial strain.

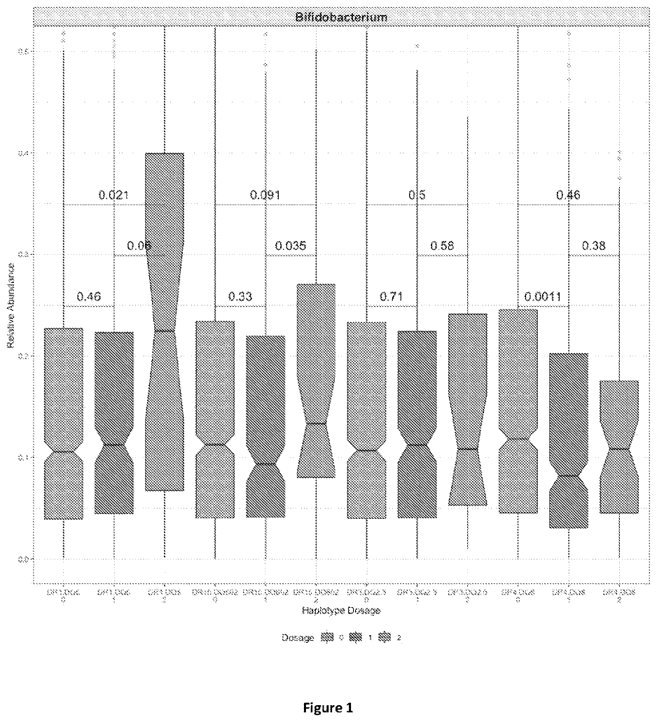

To exemplify the impact HLA plays on the gut microbiome, the core microbiomes of individuals with the highest genetic protection from and risk for autoimmune diseases (homozygous HLA haplotypes DR15-DQ6.2, DR3-DQ3.5, and DR4-DQ8) were compared and showed distinct population differences at 75% prevalence (FIG. 1). Dissimilarities are seen along the x-axis between protective genotype homozygous DR15-DQ6.2 and risk genotype homozygous DR3-DQ2.5. Further dissimilarities are seen along the y-axis between risk genotype homozygous DR4-DQ8 and both the protective and other risk genotype.

To focus on HLA haplotypes impact, Kruskal-Wallis one-way analysis of variance was used to determine confounding variables from the metadata that impacted total abundance and relative abundance of Bifidobacterium (Table 2). The most significant according to p-value and false discovery rate (FDR) were months of total breastfeeding binned into three categories (1-3 months, 4-7 months, and 8-9 months), the presence or absence of siblings at birth, having a single mother, and possessing the haplotype HLA DR4-DQ8 or just DQ8. The relative abundance of Bifidobacterium was the highest with 8-9 months of breastfeeding compared to 1-3 months and 4-7 months. The presence of siblings increased relative abundance of Bifidobacterium, while having a single mother and or HLA DR4-DQ8 or just DQ8 decreased it.

TABLE 2
Metadata variables impacting Bifidobacterium total abundance
and relative abundance.

| | P-value | FDR | P-value | FDR |
|---|---|---|---|---|
| Variable | Reads | | Relative Abundance | |
| Total Breastfeeding | 0.0001116 | 0.000223 | 0.002003 | 0.0040068 |
| Siblings at birth | 0.0007643 | 0.001528 | 3.71E−08 | 7.43E−08 |
| DR4.DQ8 | 0.0198632 | 0.039726 | 0.000845 | 0.0016905 |
| DQ8 | 0.0198632 | 0.039726 | 0.000845 | 0.0016905 |
| Single Mother | 0.0266072 | 0.053214 | 0.024864 | 0.0497279 |

Individuals were matched on total breastfeeding, siblings at birth, and single mother due to specific interest in investigating the role of HLA on Bifidobacterium composition. Individuals homozygous for a specific haplotype were matched on these criteria to individuals with 0 instances of that haplotype to create the matched 0 dosage group, and homozygous individuals were then matched on the same criteria to individuals that were heterozygous for that haplotype, creating the matched 1 dosage group. (Table 3).

TABLE 3
Number of Individuals with Each Haplotype
Studied Before and After Matching.

| Haplotype Dosage | 0 | 1 | 2 | Total |
|---|---|---|---|---|
| Original DR1-DQ5 | 1019 | 271 | 36 | |
| Matched DR1-DQ5 | 973 | 260 | 36 | 1269 |
| Original DR15-DQ6.2 | 1014 | 273 | 39 | |
| Matched DR15-DQ6.2 | 935 | 250 | 39 | 1224 |
| Original DR3-DQ2.5 | 975 | 329 | 31 | |
| Matched DR3-DQ2.5 | 932 | 301 | 31 | 1264 |
| Original DR4-DQ8 | 900 | 379 | 59 | |
| Matched DR4-DQ8 | 869 | 355 | 59 | 1280 |

Matched on siblings at birth, total breastfeeding binned, and single mother according to haplotype dosage.
0 = no instance of haplotype, 1 = heterozygous for haplotype, 2 = homozygous for haplotype.

### Example 3—Bifidobacterium Abundance, Prevalence, and Relative Abundance

Due to an increase in average relative abundance associated with protective and neutral alleles and a decrease in average relative abundance associated with risk alleles, further analysis of downstream Bifidobacterium taxa was assessed following matching. One-year-old infants were grouped based on the absence of, heterozygous, or homozygous presence of HLA haplotypes of interest. The average relative abundance, total abundance, and prevalence of Bifidobacterium genus, species, and ASVs were compared based on mean and z-score. Overall trends between neutral and protective (DR1-DQ5 and DR15-DQ6.2) and risk (DR3-DQ2.5 and DR4-DQ8) haplotypes was observed.

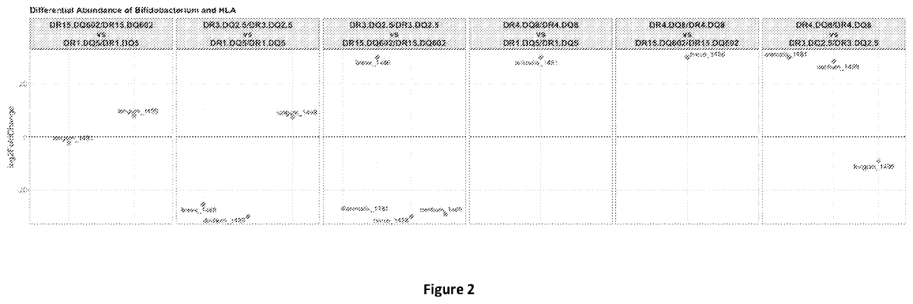

Bifidobacterium average relative abundance was the highest in both DR1-DQ5 and DR15-DQ6.2 homozygote populations and a positive correlation of increasing Bifidobacterium with increasing DR1-DQ5 haplotype dosage was clearly defined (FIG. 2A). The trend of increased relative abundance in neutral and protective homozygote groups was seen in B. adolescentis and B. pseudocatenulatum as well. Risk allele dosages impacted Bifidobacterium species and ASV differently depending on whether the population had DR3-DQ2.5 or DR4-DQ8. This difference was notable in B. animalis, B. bifidum, B. dentium, and B. longum. Also, allele dosages correlated differently with ASVs within species groups. B. longum and B. breve ASVs, specifically, showed individualized allele-dosage correlations. For example, the highest average relative abundance of B. breve 1488 was found in the DR4-DQ8 homozygote population despite a low average relative abundance of B. breve 1486. The disagreement on ASV average relative abundances were also seen in between B. longum 1491 and B. longum 1493, which held across total abundance and prevalence.

Total abundance by qPCR represents the number of bacterial DNA reads per gram of stool sample. Bifidobacterium mean total abundance decreased with increasing dosage of all haplotypes, excluding those heterozygous for DR15-DQ6.2 and DR3-DQ2.5. This same trend was seen in B. bifidum, B. longum, and B. pseudocatenulatum. The total abundance in most species and ASVs were lowest for homozygotes of all haplotypes, except B. adolescentis and B. dentium and their corresponding ASVs. B. adolescentis and ASV had highest mean abundance in DR1-DQ5 and DR3-DQ2.5 homozygotes. B. dentium and ASVs had highest mean abundance in DR4-DQ8 homozygotes. B. dentium's increased mean in those homozygous for DR4-DQ8 was seen across relative abundance and prevalence as well. However, B. dentium ASV 1489 and 1490 were not synchronized in response to dosage.

Prevalence represents the proportion of the population with the taxon of interest. Bifidobacterium had increasing prevalence correlated with increasing dosage of all haplotype populations, notably except DR15-DQ6.2 heterozygotes. Prevalence trends for most species and ASVs did not show obvious risk versus protective association, except for B. pseudocatenulatum and B. bifidum, which were most prevalent in DR15-DQ6.2 and DR1-DQ5 homozygotes and least prevalent in both risk homozygotes, especially DR3-DQ2.5.

The variability in abundances and prevalence showed that species and strain-specific trends are impacted by the presence of HLA.

### Example 4—HLA Haplotype Dosage and Bifidobacterium Relative and Differential Abundances

To further investigate significant correlations between Bifidobacterium genus, species, and ASV and HLA haplotype dosage, pairwise-comparisons of the the median relative abundance between each haplotype dosages was analyzed. At the genus-level, both the neutral and protective haplotypes had higher relative abundances than the risk haplotypes (FIG. 2). HLA DR1-DQ5 homozygotes had significantly higher relative abundance than those without the haplotype and those heterozygote (Wilcoxon: p=0.021, p=0.06, respectively) and the DR15-DQ6.2 homozygote group had a higher relative abundance than the heterozygote group (Wilcoxon: p=0.035). The DR4-DQ8 heterozygote group had a significantly lower relative abundance than those without the haplotype (Wilcoxon: p=0.0011). These data suggested that an infant without the DR4-DQ8 haplotype and with DR1-DQ5 will have a microbiome with a higher percentage of Bifidobacterium.

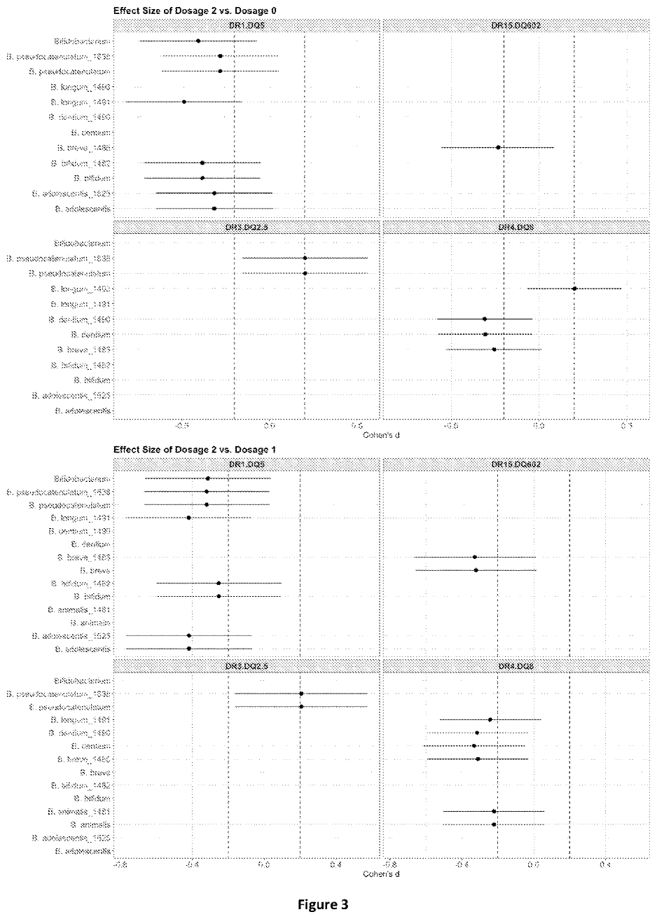

At the species level, B. longum followed a similar trend to the genus level (FIG. 3). DR1-DQ5 homozygosity correlated with higher relative abundance of B. longum compared to heterozygotes and those without the haplotype (Wilcoxon: p=0.054, p=0.04, respectively). While DR4-DQ8 heterozygotes had a significantly lower relative abundance than those without the haplotype (Wilcoxon: p=0.0032). B. pseudocatenulatum was correlated with increased protective allele dosage and decreased risk allele dosage. DR15-DQ6.2 homozygotes had B. pseudocatenulatum higher relative abundance than both the zero and single dose groups (Wilcoxon: p=0.046, p=0.05, respectively). Of note, homozygosity for risk allele DR3-DQ2.5 was correlated with higher B. breve relative abundance. DR15-DQ6.2 homozygotes had higher B. breve relative abundance than both the zero and single dose groups (Wilcoxon: p=0.0071, p=0.012, respectively).

The differential abundance was calculated to further compare levels of Bifidobacterium ASVs in homozygote HLA haplotype groups. Log-fold change was determined based on total abundance (reads/g). The ASVs with significant (DESeq2: padj<0.05) differential abundances are shown (FIG. 2). ASV differential abundances exemplify the contrasting behavior of strains within the same species. B. breve 1486 is differentially abundant both risk homozygote groups compared to the protective homozygotes DR15-DQ6.2; while B. breve 1488 is differentially abundant in the neutral and protective homozygote groups compared to risk homozygotes DR3-DQ2.5. This indicates that strain level differences may alter specific Bifidobacterium ability to proficiently colonize the gut depending on HLA haplotype makeup.

### Example 5—Effect Size of HLA Haplotype Dosage

Cohen's d effect size is a representation of the standard mean difference. Similar to the way medication dosage may be studied, using effect size in this circumstance shows the size of the impact HLA haplotype dosage has on Bifidobacterium composition within the gut microbiome. Traditionally, an effect size of above 0.2 is a small effect, between 0.5 and 0.8 is a medium effect, and anything greater than 0.8 has a large effect. Shown in FIG. 4, taxa below 0 effect size are showing correlation with the first haplotype dosage listed, while points above zero are correlated with the second dosage listed. Points outside the dotted lines are considered to have significant effect (d>0.2).

Significant effects were seen when comparing two HLA haplotype doses to zero doses and two doses to one dose, but no significant effects were seen when comparing one dose to zero doses. HLA DR1-DQ5 homozygosity had the largest effect on Bifidobacterium at the genus, species, and ASV level. Two allelic doses of HLA DR1-DQ5 compared to zero doses effected Bifidobacterium (d=0.41), B. bifidum (d=0.38), B. adolescentis (d=0.32), B. pseudocatenulatum (d=0.28), B. longum 1491 (d=0.49), B. bifidum 1482 (d=0.38), B. adolescentis 1625 (d=0.32), and B. pseudocatenulatum 1638 (d=0.28).

Possessing two neutral or protective alleles had a greater effect on Bifidobacterium abundance than having risk alleles. For example, not having DR3-DQ2.5 effected B. pseudocatenulatum and its corresponding ASV abundance (d=0.20, d=0.20, respectively). This suggests that the presence of protection-associated HLA haplotypes is more likely to have to an effect on the promotion of Bifidobacterium colonization than risk-associated haplotypes.

### DISCUSSION

Strain-level Bifidobacterium colonization is impacted by an individual's HLA composition. Based on analysis of the infant gut microbiome in a general population cohort, variability in HLA genetics is proposed as a mechanism for the failure of many probiotic clinical trials for autoimmune diseases. HLA genetics should be considered when designing effective probiotics, particularly for those at high genetic risk for autoimmune diseases.

When the number of haplotypes an individual carries of a certain HLA haplotype, be it one as heterozygous or two as homozygous, is considered in terms of haplotype dosage, the impact of HLA dosage on gut Bifidobacterium composition is clear. Bifidobacterium relative abundance increased with increasing dosage of DR1-DQ5, and a two-allele dosage had the largest effect on relative abundance, resulting in significantly higher levels of Bifidobacterium. The impact which a double dosage of DR1-DQ5 had on permitting increased levels of Bifidobacterium continued from genus to strain. HLA DR1-DQ5 may act as a protective haplotype for autoimmune diseases in many individuals as it increases gut Bifidobacterium levels.

Bifidobacterium pseudocatenulatum has previously been recommended as a potential probiotic for colitis patients following reassuring results in colitis-induced mice [58]. Since B. pseudocatenulatum relative abundance specifically increased with increasing dosage of DR1-DQ5 and DR15-DQ6.2 but decreased with increasing dosage of DR3-DQ2.5 and DQ4-DQ8, this work suggests that high-risk HLA haplotypes for autoimmune diseases may reduce gut health by reducing gut Bifidobacterium numbers and relative abundance.

The indication through differential abundance data that many Bifidobacterium strains are significantly more abundant in individuals not homozygous for HLAs of interest suggests that having a double dose of certain HLA may restrict productive colonization of certain Bifidobacterium strains.

These results have three significant implications: 1) certain class II HLA haplotypes may serve as bacterial gatekeepers early in life, clearing beneficial microbes, and creating the potential for dysbiosis and inflammation; 2) gut microbial dysbiosis and inflammation during infancy derived from host HLA genetics may be a common precedent to autoimmune diseases; and 3) HLA gatekeeping may prevent gut colonization of beneficial bacteria in genetically at-risk individuals who could most benefit from probiotic therapy. If certain class II HLA polymorphisms are serving as bacterial gatekeepers early in life, this could indicate that individuals with risk-associated alleles may be inherently prone to gut dysbiosis and may not be able to benefit from standard probiotics. In other words, those who need the benefit of efficacious probiotics the most are genetically prevented from obtaining their benefit of many, but not all, Bifidobacterium strains. A prospective, general-population cohort permits the discovery of correlations between HLA genetics and Bifidobacterium colonization levels. The ABIS general-population cohort provides a unique repository from which to reveal bacterial strains that can serve as effective probiotics in communities with and without autoimmune-associated risk alleles.

### REFERENCES

[1] Russell et al., Nature Communications. 10 (2019) 3621.
[2] Andeweg et al., MSphere. 6 (n.d.) e00476-21.
[3] Hall et al., Nat Rev Genet. 18 (2017) 690-699. doi.org/10.1038/nrg.2017.63.
[4] Davis-Richardson et al., Diabetologia. 58 (2015) 1386-1393.
[5] Davis-Richardson et al., Front. Microbiol. 5 (2014).
[6] Murri, et al., BMC Med. 11 (2013) 46.
[7] Abualrous, et al., Current Opinion in Immunology. 70 (2021) 95-104.
[8] Erlich, et al., Diabetes. 57 (2008) 1084-1092.
[9] Noble,et al., Cold Spring Harb Perspect Med. 2 (2012) a007732.
[10] Noble, et al., Curr Diab Rep. 11 (2011) 533-542.
[11] Jerram, et al., The Genetic Architecture of Type 1 Diabetes, Genes (Basel). 8 (2017).
[12] Rappazzo, et al., Nat Commun. 11 (2020).
[13] Kubinak, et al., Nat Commun. 6 (2015).
[14] Koyama, et al., Immunity. 51 (2019) 885-898.e7.
[15] Wosen, et al., Front Immunol. 9 (2018).
[16] Sarri, et al., Immunol Lett. 238 (2021) 75-95.
[17] Atkinson, et al., Lancet. 383 (2014) 69-82.
[18] Goyette, et al., Nat Genet. 47 (2015) 172-179.
[19] Bodis, et al., Rheumatol Ther. 5 (2018) 5-20.
[20] Endesfelder, et al., Microbiome. 4 (2016) 17.
[21] Moher et al., Syst Rev. 4 (2015) 1.
[22] Ludvigsson, et al., Pediatr Diabetes. 2 (2001) 170-174.
[23] Ilonen, et al., Pediatric Diabetes. 17 (2016) 8-16.
[24] Russell, et al., Sci Rep. 11 (2021) 1943.
[25] Milletich, et al., ABIS study., Front. Microbiol. (2022).
[26] Pruesse, et al., Nucleic Acids Res. 35 (2007) 7188-7196. doi.org/10.1093/nar/gkm864.
[27] Quast, et al., Nucleic Acids Res. 41 (2013) D590-596.
[28] McMurdie, et al., PLoS One. 8 (2013) e61217.
[33] Love, et al., Biology. 15 (2014) 550.
[34] Groele, et al., BMJ Open Diabetes Res Care. 9 (2021) e001523.
[35] Savilahti, et al., Diabetologia. 61 (2018) 2668-2670.
[36] Bianchini, et al., Hum Vaccin Immunother. 16 (2019) 86-94.
[37] H\dot{a}kansson, et al., Nutrients. 11 (2019) 1925.
[38] Smecuol,et al., Benef Microbes. 11 (2020) 527-534.
[39] Francavilla, et al., J Clin Gastroenterol. 53 (2019) e117-e125.
[40] Klemenak, et al., Dig Dis Sci. 60 (2015) 3386-3392.
[41] Savilahti, et al., Journal of Pediatric Gastroenterology and Nutrition. 66 (2018) 937-940.
[42] Olivares, et al., Br J Nutr. 112 (2014) 30-40.
[43] Bjarnason, et al., Inflammopharmacology. 27 (2019) 465-473.
[44] Altun, et al., Turk J Gastroenterol. 30 (2019) 313-320.
[45] Matsuoka, et al., Dig Dis Sci. 63 (2018) 1910-1919.
[46] Palumbo, et al., Biomed Pap Med Fac Univ Palacky Olomouc Czech Repub. 160 (2016) 372-377.
[47] Tamaki, et al., Dig Endosc. 28 (2016) 67-74.
[48] Yoshimatsu, et al., World J Gastroenterol. 21 (2015) 5985-5994.
[49] Groeger, et al., Gut Microbes. 4 (2013) 325-339.
[50] Wildt, et al., J Crohns Colitis. 5 (2011) 115-121.
[51] Mandel, et al., Complement Altern Med. 10 (2010) 1.
[52] Cannarella, et al., Nutrition. 89 (2021) 111282.
[53] Sartor, et al., Gastroenterology. 126 (2004) 1620-1633.
[54] Di Cagno,et al., BMC Microbiology. 11 (2011) 219.
[55] Henrick, et al., Pediatr Res. 86 (2019) 749-757.
[56] Henrick, et al., Cell. 184 (2021) 3884-3898.e11.
[57] Chen, et al., J Agric Food Chem. 69 (2021) 1496-1512.

What is claimed is:

1. A method comprising
(a) identifying the presence of a protective HLA haplotype allele in a sample from a subject; and
(b) administering a composition comprising Bifidobacterium genus probiotic to the subject.

2. The method of claim 1, wherein the protective HLA haplotype allele comprises HLA-DR15/DQ6.2.

3. The method of claim 1 or claim 2, wherein the subject is homozygous for the protective HLA haplotype allele.

4. The method of claim 1 or claim 2, comprising identifying that the subject is homozygous for HLA-DR15/DQ6.2.

5. The method of any one of 1-4, wherein the composition comprises B. pseudocatenulatum.

6. The method of any one of 1-5, wherein the composition comprises B. pseudocatenulatum ASV 1638 (SEQ ID NO: 1).

7. The method of claim 1 or claim 2, wherein the method further comprises identifying the presence of a non-protective HLA haplotype allele in a sample from the subject.

8. The method of claim 7, wherein the non-protective HLA haplotype allele comprises HLA-D3/DQ2.5 or HLA-DR4/DQ8.

9. The method of claim 1 or claim 2, wherein the subject is heterozygous for the protective HLA haplotype allele.

10. The method of claim 9, comprising identifying that the subject is heterozygous for (a) HLA-D3/DQ2.5 or (b) HLA-DR4/DQ8.

11. The method of any one of claims 1-10, wherein the subject is suffering from an autoimmune disorder.

12. The method of any one of claims 1-10, wherein the subject is at risk of developing an autoimmune disorder.

13. The method of claim 11 or claim 12, wherein the autoimmune disorder is ulcerative colitis, Crohn's disease, irritable bowel syndrome, type 1 diabetes, celiac disease, or rheumatoid arthritis.

14. The method of any one of claims 1-13, wherein the sample is a stool sample.

15. The method of any one of claims 1-14, wherein the Bifidobacterium probiotic comprises B. pseudocatenulatum, B. bifium, B. breve and B. longum.

16. A method comprising
(a) identifying the presence of a non-protective HLA haplotype allele in a sample from a subject; and
(b) administering a composition comprising a Bifidobacterium selected from B. longum, B. breve and B. pseudocatenulatum to the subject.

17. The method of claim 16, wherein the non-protective HLA haplotype allele comprises HLA-D3/DQ2.5 or HLA-DR4/DQ8.

18. The method of claim 16 or claim 17, wherein the subject is homozygous for the non-protective HLA haplotype allele.

19. The method of any one of claims 16-18, wherein the subject is homozygous for HLA-DR3-DQ2.5 or HLA-DR4-DQ8.

20. The method of any one of claims 15-18, wherein the subject is homozygous for HLA-DR3-DQ2.5.

21. The method of claim 19, comprising administering a composition comprising B. longum ASV1491 (SEQ ID NO: 2) to the subject.

22. The method of any one of claims 16-19, wherein the subject is homozygous for HLA-DR4-DQ8.

23. The method of claim 22, comprising administering a composition comprising B. breve ASV 1485 (SEQ ID NO: 3) to the subject.

24. A method of preparing a probiotic composition for a subject in need thereof comprising
(a) identifying the presence of a HLA-D3-DQ2/DR3-DQ2 HLA haplotype allele in a sample from a subject; and
(b) combining B. longum ASC1491 (SEQ ID NO: 2) with a carrier, excipient or diluent.

25. A method of preparing a probiotic composition for a subject in need thereof comprising
(a) identifying the presence of a HLA-D4-DQ8/HLA-DR4-DQ8 HLA haplotype allele in a sample from a subject; and
(b) combining B. breve ASV 1485 (SEQ ID NO: 3) with a carrier, excipient or diluent.

26. A method of preparing a probiotic composition for a subject in need thereof comprising
(a) identifying the presence of a HLA-DR15-DQ6.2/HLA-DR15-DQ6.2 HLA haplotype allele in a sample from a subject; and
(b) combining B. pseudocatenulatum ASV 1638 (SEQ ID NO: 1) with a carrier, excipient or diluent.

27. A method of preparing a probiotic composition for a subject in need thereof comprising
(a) identifying the presence of a HLA-DR1-DQ5/HLA-DR1-DQ5 HLA haplotype allele in a sample from a subject; and
(b) combining B. bifidum ASV1482 (SEQ ID NO: 4) with a carrier, excipient or diluent.


SEQUENCE LISTING (4 sequences)

SEQ ID NO: 1 - DNA, 411 bp, Bifidobacterium pseudocatenulatum
  source 1..411: mol_type = other DNA
tggggaatattgcacaatgggcgcaagcctgatgcagcgacgccgcgtgcgggatgacggccttcgggttgtaaaccgcttttgatcgggagcaagccttcgggtgagtgtacctttcgaataagcaccggctaactacgtgccagcagccgcggtaatacgtagggtgcaagcgttatccggaattattgggcgtaaagggctcgtaggcggttcgtcgcgtccggtgtgaaagtccatcgcttaacggtggatctgcgccgggtacgggcgggctggagtgcggtaggggagactggaattcccggtgtaacggtggaatgtgtagatatcgggaagaacaccaatggcgaaggcaggtctctgggccgttactgacgctgaggagcgaaagcgtggggagcgaacagg

SEQ ID NO: 2 - DNA, 409 bp, Bifidobacterium longum
  source 1..409: mol_type = other DNA
tggggaatattgcacaatgggcgcaagcctgatgcagcgacgccgcgtgagggatggaggccttcgggttgtaaacctcttttatcggggagcaagcgagagtgagtttacccgttgaataagcaccggctaactacgtgccagcagccgcggtaatacgtagggtgcaagcgttatccggaattattgggcgtaaagggctcgtaggcggttcgtcgcgtccggtgtgaaagtccatcgcttaacggtggatccgcgccgggtacgggcgggcttgagtgcggtaggggagactggaattcccggtgtaacggtggaatgtgtagatatcgggaagaacaccaatggcgaaggcaggtctctgggccgttactgacgctgaggagcgaaagcgtggggagcgaacagg

SEQ ID NO: 3 - DNA, 414 bp, Bifidobacterium breve
  source 1..414: mol_type = other DNA
tggggaatattgcacaatgggcgcaagcctgatgcagcgacgccgcgtgagggatggaggccttcgggttgtaaacctcttttgttagggagcaaggcactttgtgttgagtgtacctttcgaataagcaccggctaactacgtgccagcagccgcggtaatacgtagggtgcaagcgttatccggaattattgggcgtaaagggctcgtaggcggttcgtcgcgtccggtgtgaaagtccatcgcttaacggtggatccgcgccgggtacgggcgggcttgagtgcggtaggggagactggaattcccggtgtaacggtggaatgtgtagatatcgggaagaacaccaatggcgaaggcaggtctctgggccgttactgacgctgaggagcgaaagcgtggggagcgaacagg

SEQ ID NO: 4 - DNA, 411 bp, Bifidobacterium bifidum
  source 1..411: mol_type = other DNA
tggggaatattgcacaatgggcgcaagcctgatgcagcgacgccgcgtgagggatggaggccttcgggttgtaaacctcttttgtttgggagcaagccttcgggtgagtgtacctttcgaataagcgccggctaactacgtgccagcagccgcggtaatacgtagggcgcaagcgttatccggatttattgggcgtaaagggctcgtaggcggctcgtcgcgtccggtgtgaaagtccatcgcttaacggtggatctgcgccgggtacgggcgggctggagtgcggtaggggagactggaattcccggtgtaacggtggaatgtgtagatatcgggaagaacaccgatggcgaaggcaggtctctgggccgtcactgacgctgaggagcgaaagcgtggggagcgaacagg